(19) United States
(12) Patent Application Publication
(10) Pub. No.: US 2018/0093059 A1
(43) Pub. Date: Apr. 5, 2018

(54) IMPROVED SIDESTREAM OR MAINSTREAM OROPHARYNGEAL OR NASOPHARYNGEAL CANNULA

(71) Applicant: Deltamedics, Gouvieux (FR)

(72) Inventor: Jean-Christophe Ozenne, Gouvieux (FR)

(73) Assignee: Deltamedics, Gouvieux (FR)

(21) Appl. No.: 15/560,143

(22) PCT Filed: Mar. 18, 2016
(86) PCT No.: PCT/EP2016/056004

§ 371 Date: Sep. 20, 2017

(30) Foreign Application Priority Data
Mar. 20, 2015 (FR) 1552339

(51) Int. Cl. A61M 16/04

(52) U.S. Cl. CPC A61M 16/0461; A61M 16/0475; A61M 16/0486; A61M 16/085; A61M 16/0495; A61M 2202/0208; A61M 16/0493

(57) ABSTRACT

An oropharyngeal or nasopharyngeal cannula (1) comprising a body (2) having a curved tubular portion (4) to be inserted into the mouth or a nostril of a patient and a flange (3) suitable for resting on the lips or on the base of the nostril of the patient, the cannula comprising a main conduit (5) and at least first and second auxiliary conduits (6, 7) formed in the body and extending through the flange to the main conduit, said cannula comprising, on a middle part (42) of the tubular portion, a series of peripheral openings (78) for opening the second auxiliary conduit on the outside of the tubular portion, thus making it possible to inject oxygen into the buccal or nasal cavity in order for it to be heated and humidified by the mucosal structures.

The invention concerns a device for freeing up the airways of a patient, more particularly an oropharyngeal or nasopharyngeal cannula enabling an injection of oxygen as well as a capnographic measurement.

A cannula is a straight or curved tube, flexible or rigid, allowing the passage of a fluid such as air or a liquid through an orifice.

An oropharyngeal or nasopharyngeal cannula is used in medicine to hold open the airways of an unconscious patient, such as a patient under anesthesia or a patient in a coma, and more particularly to maintain the permeability of the respiratory pathways with the hypopharynx and to facilitate the elimination of tracheobronchial secretions.

A nasopharyngeal cannula is generally in the form of a flexible tube of plastic material comprising two portions.

A first portion is slightly curved and flexible, or semi-rigid, so as to follow the course of the nasal cavity as far as the pharynx, and thus free up a channel as far as the pharynx. The free end of the first portion comes to be placed behind the base of the tongue, on top of the epiglottis. In this way, an air passage to the lungs is maintained.

A second portion comprises a flange which rests against the entrance of the nostril of the patient once the cannula is in place and which prevents any excessive advancement of the cannula.

As for an oropharyngeal cannula, this is generally in the form of a Guedel or Berman cannula. The Guedel cannula is formed by a semi-rigid tube of plastic material comprising two parts, a tube and a flange.

A first portion of the tube is curved so as to follow the path of the buccal cavity as far as the pharynx. Its shape in particular makes it possible to move forward the tongue mass, moving it away from the posterior wall of the pharynx, and thus freeing up a channel between the tongue and the palate of the patient as far as the pharynx. In this way, an air passage is maintained to the lungs, and the tongue is held in position so that it does not droop backward onto the epiglottis and obstruct the respiratory pathways, especially when the unconscious patient is lying on their back.

A second portion of the tube is straight and reinforced on the inside by a rigid ring designed to lie in the region of the dental zone. The rigidity of this zone makes it possible to keep the orifice open when the cannula is placed between the teeth, preventing an intubated patient from obstructing an orotracheal tube by biting it, for example.

The flange is designed to rest against the lips of the patient once the cannula is in place.

There are many sizes of oropharyngeal or nasopharyngeal cannula. The sizes vary between children and adults. Their size is defined primarily as a function of the internal diameter of the cannula. For the oropharyngeal cannulas, the sizes likewise vary as a function of the distance between the labial commissure and the angle of the jawbone in children and adults. In adults, one may also use the distance between the labial commissure and the ear lobe.

During an operation under anesthesia, a facial mask is generally used to inject oxygen into the patient via the conduit of the nasopharyngeal cannula. The use of a mask, however, interferes with access to the face of the patient in case of need.

What is more, the use of a mask involves a complicated placement in order to be able to perform a capnography of the air expired by the patient and this capnography is often imprecise.

Capnography is a measuring of the concentration or partial pressure of carbon dioxide in the air expired by a patient. Such measurements are often used in patients under anesthesia. The presence of carbon dioxide in the air expired in several expirations by a patient just intubated in particular allows a confirmation that the endotracheal tube is properly placed in the trachea.

Capnography furthermore makes it possible to obtain an indirect measurement of the partial pressure of carbon dioxide in the arterial blood. This information makes it possible to evaluate the circulatory status of the patient. Capnography reflects directly the capacity of elimination of carbon dioxide by the lungs of the patient, and indirectly the production of carbon dioxide by the tissues and its transport to the lungs.

It makes it possible to detect at a very early stage the signs of respiratory deficiency, such as hypoventilation, or a disconnection of a circuit or a tube in the esophagus. During an operation under anesthesia, capnography is able to provide information such as the frequency and regularity of ventilation, which is more useful than that furnished by an oxygen meter.

It provides a method of rapid detection of critical conditions, such as a tracheal tube wrongly placed, a fault in the ventilation, a circulatory defect, and of preventing irreversible complications.

An adapted nasopharyngeal device is known from document US 2007/0095347 for performing both an injection of oxygen and a sampling of the gases expired by the patient to perform a measurement of the level of carbon dioxide free from the use of a cumbersome facial mask.

However, such a device does not use a nasopharyngeal cannula, even though the respiratory pathways may be obstructed, which does not guarantee an optimal injection of oxygen and a sampling of carbon dioxide in the best of conditions.

Moreover, the device so implemented is on the one hand heavy and very bulky in the area of the nasal zone of the patient, and on the other hand it has a complicated hook-up to the patient.

There is furthermore known from document US 2008/0000481 an oropharyngeal device of adapted form to be inserted into the mouth of a patient. The device comprises a body having at least two channels extending in the body to form two air passages through which it is possible to inject oxygen, on the one hand, and extract carbon dioxide, on the other hand. The channels are formed in the body of the device, which comprises a flange at the proximal portion to prevent the device from being inserted too deeply into the mouth.

These devices, which are solely oropharyngeal, do not have simple and rapid means of fixation on the patient to allow a holding on the patient once the device has been installed in the mouth of the patient, while allowing a rapid extraction of the device in the event of the patient awakening or in the event that an intubation is necessary.

There is likewise known from documents US 2007/267024, US 2008/308108, U.S. Pat. No. 4,821,715 and EP 1 188 457 an oropharyngeal or nasopharyngeal device adapted to form a passage for gas to be inhaled, in which there is situated a pair of conduits adapted to be slid into the internal passage(s) for injecting of oxygen, in particular at a proximal portion, and to remove the gas expired, especially on a distal portion, in order to measure the level of CO₂.

Neither do these devices have simple and rapid means of fixation on the patient allowing them to be held on the patient once the device has been installed in the mouth or the nose of the patient, while enabling a rapid extraction of the device in the event of the patient awakening or in the event that an intubation is necessary.

These devices likewise have the drawback of containing a plurality of pieces, one piece being composed of the cannula and one piece being composed of the injection and extraction tubes to be inserted. The additional time for the assembly process may be critical especially in an emergency situation.

An oropharyngeal or nasopharyngeal cannula needs to be installed and secured on the patient so that it can be removed when needed, especially in event of the patient awakening. The fixation of the cannula makes it possible to maintain the device in position, yet this fixation should be easily removable.

An oropharyngeal cannula such as a Guedel cannula may cause vomiting in a conscious patient. A patient returning to consciousness will spontaneously spit out the cannula when the cough reflex returns to him. It may also be replaced at any time by an intubation device.

A nasopharyngeal cannula is most often put in place during the post-anesthesia recovery period to facilitate the bronchial clearance, and also in cases where the patient is semi-conscious. Although a nasopharyngeal cannula is generally better tolerated by a patient than an oropharyngeal cannula of Guedel type, some patients may experience more or less pronounced difficulty which may result in the removal of the nasopharyngeal cannula in an emergency.

There is also known from document U.S. Pat. No. 3,802,431 a nasal cannula for injection of oxygen in the nostrils of the patient. The device is composed of a small cannula placed at the entrance of each nostril for injecting oxygen into each nostril with the aid of tubes connected to the cannulas and flexible enough to be passed behind the ears of the patient once the device is in place on the patient.

However, such a device does not have a nasopharyngeal cannula and only enables the injection of a fluid into the nasal cavity or at the entrance of the nostrils.

Furthermore, the injection of oxygen directly into the pharynx of the patient has drawbacks, in particular the drying out of the mucous membrane, which may result in its lesion.

The invention proposes to remedy the aforementioned drawbacks by providing an oropharyngeal or nasopharyngeal cannula adapted to enable an optimized injection of oxygen and a sampling of expired air to perform a capnographic measurement, and to secure the cannula in place on the patient in particular with the aid of injection/extraction tubes.

According to one aspect of the invention, there is proposed an oropharyngeal or nasopharyngeal cannula, comprising a body having a curved tubular portion to be inserted into the mouth or a nostril of a patient and a flange suitable for resting on the lips or on the base of the nostril of the patient, the cannula comprising a main conduit forming a fluidic passage between the flange and the tubular portion of the body, and two auxiliary conduits formed in the body and extending from the flange to the main conduit, the second auxiliary conduit extending at least partly in the tubular portion before emerging into the main conduit.

The base of the patient's nostril comprises the columella and the nasal wing of the nostril.

According to one general characteristic of the invention, the cannula comprises, on a median part of the tubular portion, a series of peripheral orifices made in the tubular portion at a portion opposite the second auxiliary conduit in order for the second auxiliary conduit to emerge on the outside of the tubular portion via the peripheral orifices.

The median part of the tubular portion is situated between the free end of the tubular portion and its portion integrated with the flange. The median part of the tubular portion of the oropharyngeal or nasopharyngeal cannula is the part designed to be situated in the buccal or nasal cavity once the cannula is put in place on the patient.

The peripheral orifices provided in the middle of the tubular portion make it possible to deliver oxygen not directly in the pharynx, but rather upstream, that is, in the buccal or nasal cavity. The injection of oxygen via the buccal or nasal cavity makes it possible to warm and humidify the oxygen before its aspiration into the pharynx and the following respiratory passageways. The oxygen is warmed and humidified by the mucous structures of the buccal or nasal cavity.

Moreover, by delivering at least a portion of the oxygen injected into the patient in the buccal or nasal cavity, the flow rate of oxygen leaving the auxiliary conduit toward the main conduit via an orifice which may be provided for this purpose is all the more reduced in the case when the peripheral orifices are the only exit orifices from the auxiliary conduit delivering oxygen. The flow rate so reduced makes it possible to prevent any “washing”, that is, any dilution of the carbon dioxide expired.

Advantageously, the first auxiliary conduit may extend solely in the flange and emerge at an entry orifice opposite the main conduit, and the flange may comprise an abutment projecting into the main conduit situated at the edge of an entry orifice of the first auxiliary conduit to deflect a fraction of the air expired by the patient through the main conduit toward the first auxiliary conduit through the entry orifice.

Since the abutment extends partly into the main conduit of the cannula through which the air is expired, it deflects a portion of the air expired from its exit trajectory. A portion of the air deflected may thus be deflected and captured, or even aspirated, in and by an entry orifice of the air expired, which communicates with the first auxiliary conduit.

Preferably, each auxiliary conduit emerges in the flange at an entry/exit orifice disposed radially on the flange (3) such that the entry/exit orifices are oriented laterally on either side of the mouth or the nostril of the patient when the cannula is installed in the patient.

Thus, by orienting the entry/exit orifices, the tubes for injection/extraction coupled to the entry/exit orifices extend in the direction of the ears of the patient, on either side of the nostril of the patient. In this way, each injection/extraction tube can be passed behind an ear to secure the nasopharyngeal cannula on the patient in an easily removable manner.

Preferably, for the nasopharyngeal cannula, the direction between each entry/exit orifice and the axis of the main conduit makes an angle between −10° and 20°, and more particularly between 0° and 10°, with an axis orthogonal to the nose of the patient when the cannula is installed on the patient.

An angle between −10 and 20°, especially an angle of around 0°, makes it possible to direct the injection/extraction tubes toward the upper edge of the ears, and thus facilitate the passage of the tubes behind the ears.

For the oropharyngeal cannula, the direction between each entry/exit orifice and the axis of the main conduit preferably makes an angle between 0° and 30°, and more particularly between 10° and 20°, with an axis passing through the commissures of the lips of the patient when the cannula is installed on the patient.

Preferably, each entry/exit orifice is connected to an auxiliary conduit via a portion of auxiliary conduit extending radially in the flange and making an angle between 0° and 20° and more particularly an angle of around 10° with the plane defined by the surface of the flange so as to orient the corresponding entry/exit orifice toward the patient's face.

This angle makes it possible to orient the injection/extraction tubes connected to the entry/exit orifices so as to maintain them as close as possible to the patient's face, and thus reduce the risks of them being torn out, or of an element or an implement getting tangled in the injection/extraction tubes.

The thickness of the flange should have a sufficient thickness to enable the passage of the auxiliary conduits.

The cannula may advantageously comprise furthermore at each entry/exit orifice disposed radially on the flange a projecting portion of the flange forming a sheath able to receive and hold in position the flexible injection/extraction tubes.

The two projecting portions form radial prominences on the periphery of the flange around each entry/exit orifice. These prominences, preferably rounded, make it possible to provide a sheath, or scabbard, to receive and hold the lateral injection/extraction tubes in place while minimizing the overall volume of the flange.

Advantageously, the cannula may comprise two flexible injection/extraction tubes able to be passed behind the ears of the patient when the cannula is installed on the patient, a first end of each tube being connected to an entry/exit orifice.

The connection of the tubes to the entry/exit orifices may be done in a removable manner or be integrated and immovable. The presence of integrated tubes on the oropharyngeal or nasopharyngeal cannula makes it possible to avoid any assembly process except for the connecting of the free ends of the tubes to means of extraction, such as a capnograph, or means of injection during the installation of the cannula on the patient.

The cannula may advantageously comprise a clamping ring able to hold the two tubes together and to slide along the two tubes so as to hold the cannula in place when it is installed on the patient and each tube passes behind an ear.

The clamping ring, once the tubes have been passed behind each ear, makes it possible to tighten the tube underneath the chin by moving the clamping ring up toward the chin. This makes it possible to ensure a secure fixation of the cannula while still allowing for a rapid removal of the cannula if need be.

Advantageously, a second end of the injection/extraction tubes may comprise means of connection to injection or extraction instruments.

The means of connection may be specific connectors dedicated to measurement or gas injection instruments. By having a specific connector for each tube, the risks of a wrong connection are zero, even in an emergency situation, since each connector is dedicated to a specific instrument.

Preferably, the second auxiliary conduit is devoted to the injection of oxygen in the respiratory pathways of the patient, and the first auxiliary conduit is devoted to the extraction of gases expired by the patient in order to perform a capnographic measurement. To do so, the second auxiliary conduit comprises an entry orifice connected to a tube for injection of oxygen in the respiratory pathways of the patient, and the first auxiliary conduit comprises an exit orifice connected to a tube for extraction of gases expired by the patient to perform a capnographic measurement.

Preferably, the tube for injection of oxygen is configured to deliver cooled oxygen or a cooled gas mixture, and the series of peripheral orifices is disposed in the tubular portion so as to have the orifices oriented toward the top of the buccal or nasal cavity once the cannula is inserted into the mouth or a nostril of the patient.

The use of cooled oxygen makes it possible to oxygenate the patient while cooling the brain, which allows for reducing the oxygen consumption by the brain.

In one variant, the cooling of the brain may be achieved by the injection of another cooled gas or a gas able to cool the brain of a patient.

Moreover, the possible orientation of the peripheral orifices toward the top makes it possible to maximize the space between the peripheral orifices and the wall of the buccal or nasal cavity and thus to increase the volume able to receive the cooled oxygen.

The tubular portion preferably comprises one free end and one end integrated with the flange, and the second conduit extends as far as a distal portion comprising the free end of the tubular portion.

By separating the places where the auxiliary conduits emerge in the main conduit, perturbations due in particular to the turbulence created by the injection of oxygen are reduced for the extraction of the gases expired by the patient, and the measurement of carbon dioxide involves a better signal to noise ratio.

In one variant, there may also be two tubes for sampling of carbon dioxide connected to the two entry/exit orifices, the two tubes being connected to one and the same capnography device. In this way, the device can be used only for the sampling of carbon dioxide. In this configuration, the two auxiliary conduits may emerge into the main conduit at similar or separate places. In similar fashion, the oropharyngeal or nasopharyngeal cannula may be used solely for the injection of oxygen via the two auxiliary conduits.

At least one of the two auxiliary conduits may advantageously comprise a cross section of oblong shape at least for the tubular portion of the body.

The conduits may have different cross-sectional shapes, such as a circular cross-sectional shape, for example. By providing an oblong shape for the auxiliary conduit, the volume of fluid, particularly a gas, which can be channeled in the conduit is greater than in the case of a conduit with cylindrical cross section. The oblong cross sections may be produced so as to follow the contours of the main conduit of the oropharyngeal or nasopharyngeal cannula. In this way, one obtains curved oblong cross sections, which makes it possible to produce the auxiliary conduits in the walls of the cannula without the need to increase in a significant manner the thickness of the walls of the body of the cannula. And this while preserving a significant volume of gas transfer.

Advantageously, the cannula may comprise at least one supplemental conduit formed in the body and extending from the flange to at least the tubular portion of the body. The supplemental conduit may be devoted to the aspiration of secretions in the pharynx of the patient so as to prevent the airways from being obstructed by these secretions. The aspiration of secretions can also be done via a tube inserted into the main conduit.

Advantageously, at least one of the auxiliary conduits may comprise an orifice emerging into the main conduit and having a truncated conical shape with a larger cross section than the mean cross section of the auxiliary conduit.

The truncated conical shape of the orifice emerging into the main conduit in the area of the tubular portion of the body makes it possible to promote a good distribution of the oxygen injected, on the one hand, and to optimize the sampling of carbon dioxide, on the other hand.

Preferably, the tubular portion has one free end and one end integrated with the flange, the free end of the tubular portion of the body having a rounded shape. In one variant, the free end may have a shape with rounded edges:

The rounded shape of the free end makes it possible to reduce the risks of injury during the introduction of the oropharyngeal or nasopharyngeal cannula, especially as compared to a nasopharyngeal cannula having a beveled free end.

The body may be made of a flexible and slippery plastic material. In this way, the oropharyngeal or nasopharyngeal cannula may be introduced without the use of a lubricating gel.

Other advantages and characteristics of the invention will appear from an examination of the detailed description of one embodiment, in no way limiting, and the enclosed drawings, in which:

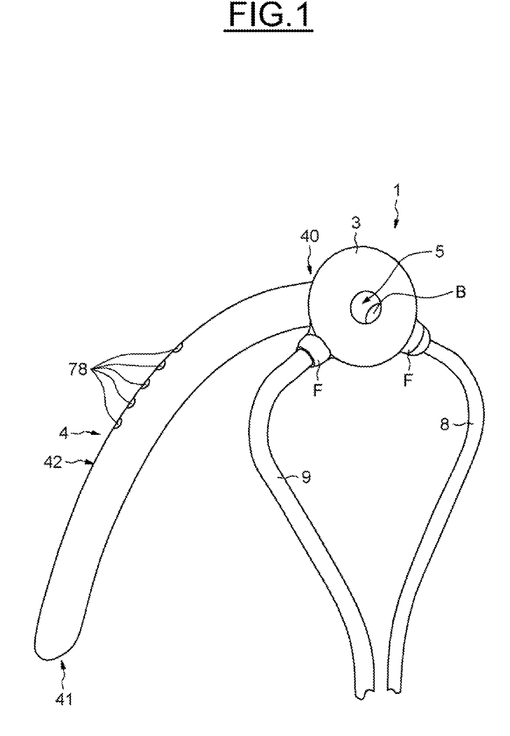

FIG. 1 illustrates a perspective view of a nasopharyngeal cannula according to one embodiment of the invention;

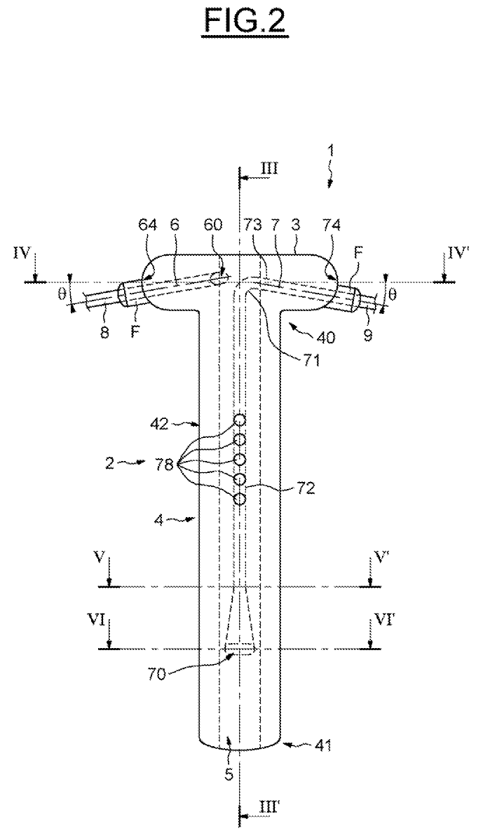

FIG. 2 represents, schematically, a top view of the nasopharyngeal cannula of FIG. 1;

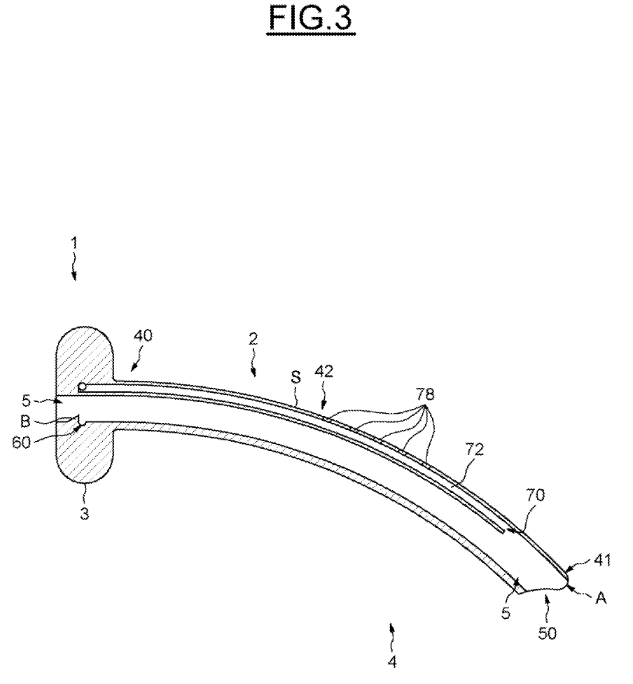

FIG. 3 illustrates a cross-sectional view in a longitudinal plane III-III′ of the nasopharyngeal cannula of FIGS. 1 and 2;

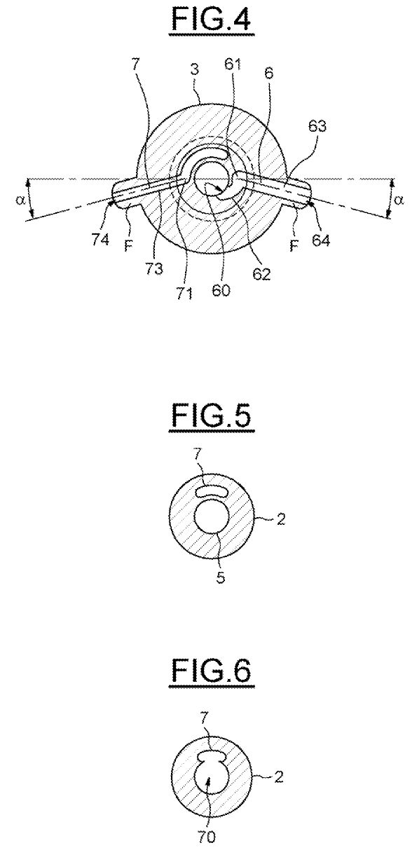

FIG. 4 illustrates a cross-sectional view in a transverse plane IV-IV′ of the nasopharyngeal cannula of FIGS. 1 and 2;

FIG. 5 illustrates a cross-sectional view in a transverse plane V-V′ of the nasopharyngeal cannula of FIGS. 1 and 2;

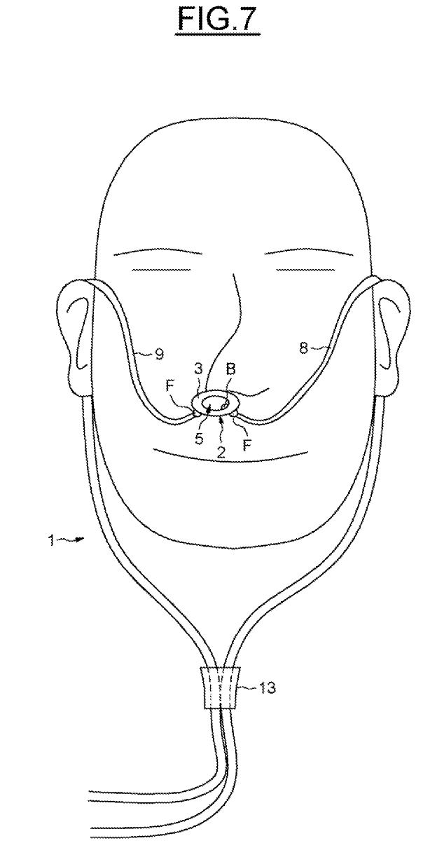

FIG. 6 illustrates a cross-sectional view in a transverse plane VI-VI′ of the nasopharyngeal cannula of FIGS. 1 and 2;

FIG. 7 presents a diagram of a nasopharyngeal cannula of FIG. 1 installed on a patient;

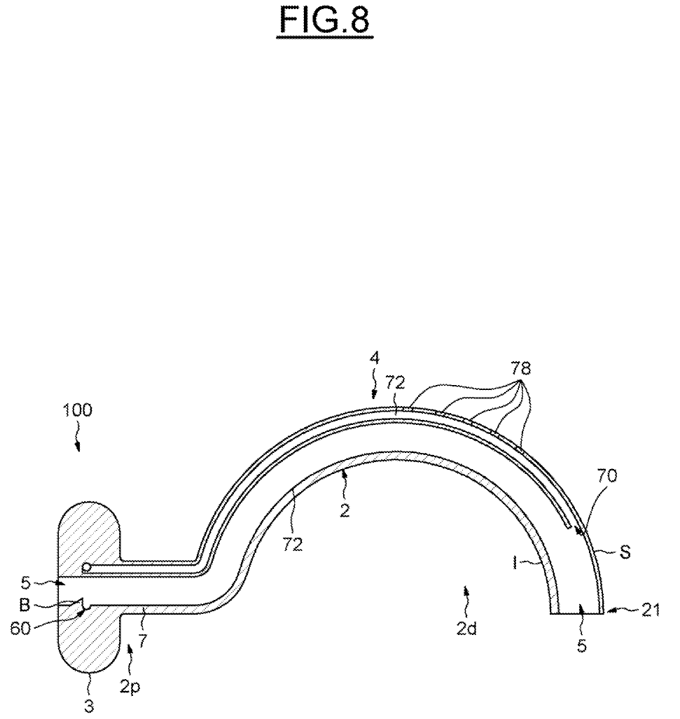

FIG. 8 presents a cross-sectional view in a longitudinal plane of an oropharyngeal cannula according to one embodiment of the invention.

FIGS. 1 and 2 show schematically a perspective view and a top view of a nasopharyngeal cannula 1 according to one embodiment of the invention.

The nasopharyngeal cannula 1 comprises a body 2 made of slippery flexible plastic material, such as medical PVC, for example, formed of a flange 3 and a tubular portion 4. As illustrated in FIG. 3 showing a longitudinal section along the plane of the cannula 1 of FIG. 2, the tubular portion 4 has a curved shape so as to allow the insertion of the nasopharyngeal cannula 1, and especially its tubular portion 4, into the nostril of a patient as far as the pharynx so as to pass behind the base of the tongue and thus maintain the pharynx open.

The flange 3 is designed to rest against the outside of the base of the nostril of the patient once the cannula 1 has been inserted into the nostril of the patient. The body 2 comprises a main conduit 5 extending from the flange 3 to the free end 41 of the tubular portion 4. The main conduit 5 thus forms a fluidic passage between the flange 3 and the tubular portion 4 of the body 2 of the nasopharyngeal cannula 1. Once the cannula 1 is installed on the patient, the main conduit 5 forms a fluidic passage between the pharynx and the nostril of the patient.

As is illustrated in FIGS. 2 and 3, the nasopharyngeal cannula 1 comprises a first auxiliary conduit 6 formed in the thickness of the body 2 separating the main conduit 5 from the outside of the body 2. The first auxiliary conduit 6 extends in the flange 3 between an entry orifice 60 and an exit orifice 64. It emerges into the main conduit 5 via the entry orifice 60, as is represented in FIGS. 2 and 4.

As represented in FIG. 2 and in FIG. 4, which shows a cross-sectional view along plane IV-IV′ of the nasopharyngeal cannula 1 of FIG. 2, the first auxiliary conduit 6 comprises a first elbow 61 joining a first portion 62 of the first auxiliary conduit 6 extending as far as the entry orifice 60 and a second portion 63 of the first auxiliary conduit 6 extending into the flange 3 as far as the exit orifice 64.

The nasopharyngeal cannula 1 comprises a second auxiliary conduit 7 likewise formed in the thickness of the body 2. The second auxiliary conduit 7 traverses the body 2 from the flange 3 and emerges into the main conduit 5 via an exit opening 70 disposed upstream from the free end 41 of the tubular portion 4, as represented in FIGS. 2 and 3 as well as in FIG. 6, which represents a view along the cross-sectional plane VI-VI′ of the nasopharyngeal cannula 1 of FIG. 2. By thus positioning the exit opening 70 upstream from the free end 41 of the tubular portion 4, one reduces the risks of obstruction of the exit opening 70 by secretions in the throat.

In order to further reduce the potential accumulation of secretions in the pharynx, the nasopharyngeal cannula 1 may comprise a supplemental conduit, not shown, formed in the body 2 and extending from the flange 3 as far as the free end 41 of the tubular portion 4. The supplemental conduit is connected to means of aspiration in order to eliminate the secretions.

As represented in FIGS. 2 to 4, the second auxiliary conduit 7 comprises a second elbow 71 joining a first portion 72 of the second auxiliary conduit 7 extending along the longitudinal plane as far as the exit opening 70 and a second portion 73 of the second auxiliary conduit 7 extending in the flange 3. The second portion 73 of the second auxiliary conduit 7 extends in the flange 3 along a radial direction as far as an entry opening 74.

The tubular portion 4 has a general rounded shape A at the free end 41. The rounded shape A makes it possible to reduce the risks of injury during the introduction of the nasopharyngeal cannula 1 in the nostril of the patient.

In order to optimize the injection or the extraction of a fluid via an auxiliary conduit 6 or 7, the first auxiliary conduit 6 and the second auxiliary conduit 7 may each have a truncated conical shape at the end emerging into the entry orifice 60 or the exit opening 70. The cross sections of the entry orifice 60 and the exit opening 70 may thus be respectively larger than the cross sections of the first and second auxiliary conduits 6 and 7. This truncated conical shape makes it possible to optimize the removal in the case of an extraction or to promote the distribution of the fluid injected into the main conduit 5. As shown in FIG. 2, the exit opening 70 of the second auxiliary conduit 7 has a truncated conical shape.

The second auxiliary conduit 7 is designed to deliver a stream of oxygen. The first auxiliary conduit 6 is designed to remove a portion of the gases expired by the patient to measure the level of carbon dioxide in the expired gases by means of a capnograph.

To do so, the exit orifice 64 and the entry opening 74 are respectively connected to a tube for extraction 8 of the air expired and a tube for injection 9 of oxygen, as is illustrated in FIG. 2. The connection is made by soldering or by overmolding of the flange 3 on the tubes for extraction and injection 8 and 9, or by means of connectors.

In the example illustrated in FIGS. 1, 2 and 4 in particular, the nasopharyngeal cannula 1 comprises at the exit orifice 74 on the one hand and the entry orifice 64 on the other hand a projecting portion F of the flange 3 at the radial periphery of the flange 3. The two sheaths F form rounded radial prominences at the periphery of the flange 3 around each entry/exit orifice 74 and 64. Each projecting portion F forms a sheath dimensioned and shaped to receive and hold in position one of the flexible tubes for injection/extraction 8 and 9. In the embodiment illustrated, the flexible tubes 8 and 9 are held in position in the sheaths F by gluing.

The free end of the injection tube 9 may comprise a specific connector, for example, a standard conical connection for oxygen, designed to be connected to a device delivering a flow of oxygen, optionally via a cross section reducer of the tube. The free end of the extraction tube 8 on the other hand may comprise a specific connector, such as a “luer lock” type connection, designed to be connected to a capnograph.

As is illustrated in FIG. 4, the second portion 63 of the first auxiliary conduit 6 and the second portion 73 of the second auxiliary conduit 7 each make an angle α with an axis passing through the first elbow 61 and the second elbow 71. The angle α has a value between −10° and 20° and preferably near 0°. In the embodiment illustrated in FIGS. 1 to 6, the angle α is near −10°. This angle allows the injection tube 9 and the extraction tube 8 to each be oriented in the direction of the upper edge of an ear of the patient so as to facilitate the passage of the extraction and injection tubes 8 and 9 behind the ears, as is illustrated in FIG. 7.

Moreover, the second portion 63 of the first auxiliary conduit 6 and the second portion 73 of the second auxiliary conduit 7 may each make an angle between 0° and 20° and more particularly around 10° with the plane defined by the surface of the flange 3 so as to orient the extraction and injection tubes 8 and 9 toward the patient's face and thus optimize the holding in position of the nasopharyngeal cannula 1. The extraction and injection tubes 8 and 9 being thus brought near the face of the patient, the risks of an element or an implement becoming entangled in the tubes are moreover reduced.

In order to deliver oxygen not directly into the pharynx, but upstream, that is, in the buccal or nasal cavity, and thus warm and humidify the oxygen before it arrives in the pharynx and the ensuing airways, the tubular portion 4 comprises at a median part 42 of the tubular portion 4 between the free end 41 and the end 40 integrated with the flange 3 a series of peripheral orifices 78. The peripheral orifices are made in the tubular portion 4 at a portion facing the second auxiliary conduit 7 in order to make the second auxiliary conduit 7 emerge on the outside of the tubular portion 4 via the peripheral orifices 78 and thus warm and humidify the oxygen thanks to the mucous structures of the buccal or nasal cavity.

In order to remove the air expired by the patient through the main conduit 5, the flange 3 comprises an abutment B extending as a projection into the main conduit 5. The abutment B is positioned on the periphery of the entry orifice 60 of the first auxiliary conduit 6 so as to have the entry orifice 60 between the abutment B and the tubular portion 4. The abutment B thus makes it possible to deflect a fraction of the air expired by the patient through the main conduit 5 toward the first auxiliary conduit 6 through the entry orifice 60.

FIG. 7 presents a diagram of a nasopharyngeal cannula 1 installed on a patient according to the embodiment of FIG. 1. The passage of the tubes for extraction and injection 8 and 9 behind the ears of the patient makes it possible to hold the nasopharyngeal cannula 1 in place on the nostril of the patient. In this embodiment, the nasopharyngeal cannula 1 moreover comprises a clamping ring 13 coupled with the extraction and injection tubes 8 and 9. The clamping ring 13 is mounted in a sliding manner along the extraction and injection tubes 8 and 9 so as to allow the ring 13 to be moved up until the injection and extraction tubes are tightened beneath the chin of the patient so that the nasopharyngeal cannula 1 is held in a secure manner in position on the patient.

In order to improve the evacuation of the moisture present in the extraction tube 8, the extraction tube may comprise a portion of several centimeters, such as 5 cm, of Nafion tube. This portion is preferably situated at 2 cm from the flange 3.

FIG. 8 shows schematically an example of an oropharyngeal cannula 100 according to one embodiment. The oropharyngeal cannula comprises the same elements as the nasopharyngeal cannula 1 illustrated in FIGS. 1 to 7, these elements being given the same reference numbers.

The invention proposed furnishes an oropharyngeal or nasopharyngeal cannula able to be installed and secured rapidly on the patient in a removable manner while being held in a secure manner in position on the patient. Moreover, the proposed oropharyngeal or nasopharyngeal cannula is made as a single piece or is preassembled so that one only needs to connect the tubes for injection/extraction to the particular instruments. This makes it possible to reduce the time for the installation and the risks of wrong connections.

1. An oropharyngeal or nasopharyngeal cannula comprising a body having a curved tubular portion to be inserted into the mouth or a nostril of a patient and a flange suitable for resting on the lips or on the base of the nostril of the patient, the cannula comprising a main conduit forming a fluidic passage between the flange and the tubular portion of the body, and at least a first and a second auxiliary conduit formed in the body and extending from the flange to the main conduit, the second auxiliary conduit extending at least partly in the tubular portion before emerging into the main conduit, characterized in that it comprises, on a median part of the tubular portion, a series of peripheral orifices made in the tubular portion at a portion opposite the second auxiliary conduit in order for the second auxiliary conduit to emerge on the outside of the tubular portion via the peripheral orifices

2. The cannula s claimed in claim 1, wherein the first auxiliary conduit extends solely in the flange and emerges at an entry orifice opposite the main conduit, and the flange comprises an abutment projecting into the main conduit situated at the edge of the entry orifice of the first auxiliary conduit to deflect a fraction of the air expired by the patient through the main conduit toward the first auxiliary conduit through the entry orifice.

3. The cannula as claimed in claim 1, wherein each auxiliary conduit emerges in the flange at an entry/exit orifice disposed radially on the flange such that the entry/exit orifices are oriented laterally, respectively on either side of the mouth or the nostril of the patient when the cannula is installed in the patient.

4. The nasopharyngeal cannula as claimed in claim 3, wherein the direction between each entry/exit orifice and the axis of the main conduit makes an angle between −10° and 20°, and more particularly between 0° and 10°, with an axis orthogonal to the nose of the patient when the cannula is installed on the patient.

5. The oropharyngeal cannula as claimed in claim 3, wherein the direction between each entry/exit orifice and the axis of the main conduit makes an angle between 0° and 30°, and more particularly between 10° and 20°, with an axis passing through the commissures of the lips of the patient when the cannula is installed on the patient.

6. The cannula s claimed in claim 5, wherein each entry/exit orifice is connected to an auxiliary conduit via a portion of auxiliary conduit extending radially in the flange and making an angle between 0° and 20° and more particularly an angle of around 10° with the plane defined by the surface of the flange so as to orient the corresponding entry/exit orifice toward the patient's face.

7. The cannula as claimed in claim 3, comprising furthermore at each entry/exit orifice disposed radially on the flange a projecting portion of the flange forming a sheath able to receive and hold in position the flexible injection/extraction tubes.

8. The cannula as claimed in claim 3, comprising two flexible injection/extraction tubes able to be passed behind the ears of the patient when the cannula is installed on the patient, a first end of each tube being connected to an entry/exit orifice.

9. The cannula as claimed in claim 8, comprising a clamping ring able to hold the two tubes together and to slide along the two tubes so as to hold the cannula in place when it is installed on the patient and each tube passes behind an ear.

10. The cannula as claimed in claim 8, wherein a second end of the injection/extraction tubes comprises means of connection to injection or extraction instruments.

11. The cannula as claimed in claim 1, wherein the second auxiliary conduit comprises an entry orifice connected to a tube for injection of oxygen in the respiratory pathways of the patient, and the first auxiliary conduit comprises an exit orifice connected to a tube for extraction of gases expired by the patient to perform a capnographic measurement.

12. The cannula as claimed in claim 11, wherein the tube for injection of oxygen is configured to deliver cooled oxygen or a cooled gas mixture, and the series of peripheral orifices is disposed in the tubular portion so as to have the orifices oriented toward the top of the buccal or nasal cavity once the cannula is inserted into the mouth or a nostril of the patient.

13. The cannula as claimed in claim 1, wherein the first auxiliary conduit comprises a cross section of oblong shape at least for the tubular portion of the body.

14. The cannula as claimed in claim 1, wherein at least one of the auxiliary conduits comprises an orifice emerging into the main conduit and having a truncated conical shape with a larger cross section than the mean cross section of the auxiliary conduit.

15. The cannula as claimed in claim 1, wherein the tubular portion as one free end and one end integrated with the flange the free end of the tubular portion of the body having a rounded shape.

16. The cannula as claimed in claim 1, wherein the body is made of a flexible and slippery plastic material.
